(19) United States
(12) Patent Application Publication
(10) Pub. No.: US 2025/0066703 A1
(43) Pub. Date: Feb. 27, 2025

(54) Microfluidic System for Reproducing Functional Units of Tissues and Organs In Vitro

(71) Applicant: Nortis, Inc., Bothell, WA (US)

(72) Inventors: Thomas Neumann, Seattle, WA (US); Anna A. Tourovskaia, Mountainlake Terrace, WA (US); Mark E. Fauver, Seattle, WA (US); Greg Kramer, Seattle, WA (US); Elizabeth McClure, Seattle, WA (US); Henning Mann, Seattle, WA (US)

(21) Appl. No.: 18/826,634

(22) Filed: Sep. 6, 2024

Related U.S. Application Data
(63) Continuation of application No. 16/025,806, filed on Jul. 2, 2018, now abandoned.
Application No. 16/025,806 is a division of application No. 14/431,015, filed on Mar. 25, 2015, now abandoned.

(51) Int. Cl. C12M 3/00; A01N 1/02; C12M 1/00; C12M 1/12; C12M 1/26; C12M 3/06; G01N 33/50

(52) U.S. Cl. CPC C12M 21/08; A01N 1/0231; A01N 1/0247; C12M 23/16; C12M 25/14; C12M 29/06; C12M 29/10; C12M 33/00; C12M 33/04; G01N 33/5085

(57) ABSTRACT

A microfluidic system including a number of microfluidic devices having a first perfusion path and a second separate perfusion path; the microfluidic devices each also having a chamber containing a matrix, where the matrix surrounds at least one void whose lumen is in fluidic connection exclusively with the first perfusion path, where the at least one void is populated with at least one cell type in such way that the cells are in direct contact with the matrix; where the matrix is in fluidic connection exclusively with the second separate perfusion path. The microfluidic devices are integrated onto a platform; and each of the microfluidic devices mimics at least a partial organ module.

### CROSS-REFERENCE TO RELATED APPLICATIONS

This application is a continuation of U.S. application Ser. No. 16/025,806, filed Jul. 2, 2018, which is divisional of U.S. application Ser. No. 14/431,015 filed Mar. 25, 2015, which is a national phase entry of International Application No. PCT/US2013/062307 filed Sep. 27, 2013, which claimed priority from U.S. Provisional Application No. 61/707,907, filed Sep. 29, 2012, and further claimed priority from co-pending U.S. Provisional Application No. 61/721,002 filed Oct. 31, 2012, the disclosures of which are all incorporated by reference.

### FIELD OF THE INVENTION

The present invention relates to methods for reproducing functional units of tissues and organs in vitro, and, more particularly, to systems including tissue-engineered microenvironments on a chip.

### BACKGROUND OF THE INVENTION

While the investment in pharmaceutical research and development has been growing exponentially, the number of new drugs approved by FDA has remained unchanged in the past 60 years. Nearly 95% of new drug candidates fail between pre-clinical and clinical phases of development, mainly due to drug-associated toxicity. Clearly, better pre-clinical drug-screening assays are needed. Currently pharmacokinetic and toxicological evaluation of drug candidates relies largely on animal test systems, which evidently show only very limited predictive value for clinical efficacy and toxicity. In addition, maintaining animal models drives up dramatically the cost of drug development. There are also a number of high-throughput two dimensional (2D) cell line models commonly used in drug development. Their predictive value is very limited, which is attributed to the loss of physiological context. More sophisticated humanized cell-based assays from 3D cell cultures to organs-on-chips are being developed that can address the limitations of 2D cell culture and minimize or potentially even completely replace animal models. In 3D cell-culture models cells are grown within 3D microenvironment that mimics structural, biochemical and mechanical aspects found in vivo. Such cultures are known to restore specific biochemical and morphological features characteristic of corresponding tissues in vivo. The examples of conventional static 3D cultures include hydrogel-incapsulated cells, “sandwich” cultures, multicellular spheroids, cells grown on microcarriers and microstructured support. While powerful these models still lack the complexity required for pharmacokinetic studies and have a number of other shortcomings: 1) limited nutrient supply and accumulation of metabolic waste products that can confound cell responses to drugs, 2) inability to mimic spatiotemporal biochemical gradients existing in vivo, 3) lack of mechanical cues such as flow, perfusion, pressure, mechanical stress, 4) difficult to probe, 5) problematic real-time imaging, and 6) biochemical analysis cannot be performed in live cells due to reaction-diffusion phenomena. Furthermore, it has not yet been possible to engineer microsystems that integrate multiple organ/tissue mimetics with active vascular conduits and barrier tissues.

As a result, there is an established, yet unmet need for less expensive, more sophisticated, more controlled systems for drug studies, vaccine development and other types of medical research. The present invention provides new and novel approaches for such controlled systems, including a system for integrating vascular cells and organ cells to reproduce functional units of tissues and organs in vitro. These and other important new teachings are evident from the specification and claims hereinbelow.

Medical research for the development of vaccines against parasitic diseases requires not only availability and access to the parasitic cells in vitro, but also the ability to model a host organism of the parasite. The human malaria parasite infects mosquitoes, which in turn act as vectors in transmitting the disease to humans. To develop and identify effective vaccines against malaria, investigators require a model system in which the malaria parasite can be studied in the context of the mosquito midgut, where it naturally lives. However, there are currently no systems available to culture the developmental insect stages of the human malaria parasite Plasmodium falciparum in vitro. Furthermore, research on the infectious stages of the malaria parasites, the sporozoites, depends on inaccurate and difficult to control methods that involve live, infected mosquitoes. Such approaches introduce many undesirable variables into the analysis. For example, live mosquitoes are not subject to controlled blood intake parameters and analysis of drug effectiveness must be performed by a relatively subjective manual examination of dissected mosquito intestines.

As a result, there is an established, yet unmet need for less expensive and more controlled systems for both an in vitro culture system to produce all parasite insect stages, including, for example, parasite mosquito stages, and a testing platform for drug and vaccine studies, vaccine development and other types of medical research related to diseases such as malaria. The present invention provides new and novel approaches for such a controlled system, including a model for reproducing functional units of the mosquito midgut system and the culture of the malaria parasite stages therein. These and other important new teachings are evident from the specification and claims here in below.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A microfluidic system for generating compartmentalized microenvironments of tissues and organs in vitro and for independently perfusing the compartments is herein disclosed. A microfluidic device includes at least a first perfusion path and a second separate perfusion path. The microfluidic device also has a chamber containing a matrix, where the matrix surrounds at least one void whose lumen is in fluidic connection exclusively with the first perfusion path, where the at least one void can be populated with at least one cell type in such way that the cells are in direct contact with the matrix and the matrix is in fluidic connection exclusively with the second separate perfusion path.

In another aspect, a method for reproducing a functional unit of an invertebrate in vitro, as a tissue-engineered microenvironment for the culture of parasites is disclosed including providing a microfluidic device having at least a first perfusion path and a second separate perfusion path, the microfluidic device also having a chamber. The chamber is filled with a matrix, where the matrix surrounds at least one void whose lumen is in fluidic connection exclusively with the first perfusion path, where the at least one void is populated with at least one cell type in such way that the cells are in direct contact with the matrix, and where the matrix is in fluidic connection exclusively with the second separate perfusion path. The at least one void is seeded with invertebrate cells and the invertebrate cells are perfused to proliferate and generate an invertebrate organ or tissue. Parasite stages are cultivated in the microenvironment to provide a testing microenvironment.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1A and FIG. 1D show examples of the two-compartment and three-compartment (single-cell tube and dual-cell tube) TEM-chips, respectively.

FIG. 1B and FIG. 1E illustrate the technical design of two TEM-chip types.

FIG. 1C and FIG. 1F schematically illustrate single and dual fluidic conduits respectively for lumenal fluid flow.

FIG. 2A shows an example of cells embedded in the matrix surrounding a microvascular-like cell tube including human brain astrocytes and pericytes embedded in the proximity of the microvascular tube consisting of Human Umbilical Vein Endothelial Cells (HUVECs).

FIG. 2B shows an example of cells embedded in the matrix surrounding a microvascular-like cell tube wherein pericytes and astrocytes are recruited to the walls of the microvascular tube.

FIG. 2C and FIG. 2D illustrate stimulated outgrowth of HUVECs.

FIG. 3A, FIG. 3B and FIG. 3C show an example of a three-compartment model showing a kidney module with a HEK293-tube and a corresponding vascular-cell tube created from HUVECs over a four-day period.

FIG. 4A-FIG. 4C show an example of a three-compartment setup including an intestine module with a cell tube generated from the HT29-cell line and a corresponding vascular-cell tube created from HUVECs.

FIG. 5A shows an example of a three-compartment model showing a liver module with a liver-cell tube generated from Hep-G2 cells and a vascular-cell tube generated with HUVECs.

FIG. 5B shows hepatocytes embedded into the matrix surrounding the blood vessel.

FIG. 6A-FIG. 6F show an example of a blood-brain-barrier model, in particular illustrating paracellular permeability across the wall of a vascular-cell tube (engineered from primary human microvascular endothelial cells) in a two-compartment device.

FIG. 7A and FIG. 7B show a reorganization of cells in the BBB model consisting of hCMEC/D3 (human brain microvascular cell line) and ECM-embedded pericytes and astrocytes (primary human brain cells).

FIG. 8A-FIG. 8F jointly show an example of a two-compartment model of tumor-endothelium interactions over a time period of 7 days. FIG. BD, FIG. 8E, and FIG. 8F show different focal planes of the same specimen to illustrate multiple sprouts growing toward the cancer cell cluster.

FIG. 9A-FIG. 9D jointly show an example of a three-compartment model of tumor-endothelium interactions.

FIG. 10A and FIG. 10B jointly show an example of cancer cell extravasation.

FIG. 11 illustrates an example of four connected TEM-chips forming a complex system with each chip representing a different organ.

FIG. 12 illustrates an example of an alternative architecture for connecting four TEM-chips, each representing a different organ, to a complex system.

FIG. 13 illustrates an example of an alternative architecture employing a plurality of many more physiological modules integrated into one circuit.

FIG. 14A-FIG. 14C show an example of a two-compartment mosquito midgut chip showing a cell tube with a mosquito 4A-3A-cell-coated tubule developing over a time period of 5 days.

FIG. 15A-FIG. 15F show examples of early stage oocysts in very preliminary culture environments.

FIG. 16 schematically shows an example of a midgut chip.

FIGS. 17A-17D show an example of enriched GFP-expressing Plasmodium falciparum ookinete 48 hrs. post-fertilization in suspension of RBC's.

FIGS. 17E-17F are examples including a cell tube of 4A-3B cells with injected GFP-expressing parasites in stages of zygotes and developing and matured ookinetes.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements and have been solely selected for ease of recognition in the drawings.

### DETAILED DESCRIPTION OF THE PREFERRED EMBODIMENTS

The examples presented herein are for the purpose of furthering an understanding of the invention. The examples are illustrative, and the invention is not limited to the example embodiments.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or combinations and/or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definitions

Generally, as used herein, the following terms have the following meanings unless the context suggests otherwise:

As used herein, “BBB” is understood to mean blood-brain barrier, formed by brain specific vascular endothelium.

As used herein, “ELISA” has its generally accepted meaning and is understood to mean enzyme-linked immunosorbent assay.

As used herein, “HUVEC” has its generally accepted meaning and is understood to mean human umbilical vein endothelial cells.

As used herein, “PDMS” has its generally accepted meaning and is understood to mean polydimethylsiloxane.

As used herein, “plurality” is understood to mean more than one. For example, a plurality refers to at least 3, 4, 5, 70, 1.000, 10,000 or more.

As used herein, “TEM” is understood to mean tissue-engineered microenvironments.

As used herein, “tissue” is defined as an ensemble of one or several similar types of cells from the same origin, together with extracellular matrix secretions, that is specialized to carry out one or more specific functions.

As used herein, “organ” means a higher level of organizational structure consisting of multiple tissues, where an organ function is only possible by the interaction of multiple tissues.

### Example Embodiments

Microfluidic devices for the generation of tissue-engineered microenvironments (TEM) have been developed by the inventors hereof. These devices contain a chamber filled with a three-dimensional matrix. The matrix contains tubular voids that can be populated with various cell types, resulting in tubular cell structures. These cell tubes are lumenally connected to fluidic channels of the devices and, thus, can be perfused with nutrient solutions, test substances, cell solutions or other fluids. Lumenal perfusion, and perfusion or diffusion through the matrix, allow for tight control of the micro-environmental conditions within the devices. Fluid pressure and shear stress are known to affect cell shape, proliferation, differentiation, and protein expression.

The fluidic devices are designed as small chips made of polydimethylsiloxane (PDMS) sandwiched between a glass plate and a polycarbonate plate. These tissue-engineered microenvironment chips (TEM-chips) are designed for generating in vitro models that reproduce the micro-architectural and functional parameters of various tissues and organs. Because the setup leads to tubular cell structures that are completely surrounded by matrix (for example gelled collagen I, fibrin, or combinations of collagen I, IV, and/or hyaluronan), direct contact of the cells with non-biological materials is prevented. Contact with tissue-derived proteins has been shown to support physiological behavior in vitro. On the other hand, contact with non-biological materials can adversely affect cellular responses.

The architecture of the TEM-chips allows for the generation of two or more tissue compartments that can be independently perfused and may be separated from one another by, for example, cellular barriers or other barriers. For example, a single tubular cell structure within a collagen matrix presents a two-compartment system, consisting of a lumenal compartment within the cell tube and an extralumenal compartment comprised by the surrounding matrix. Both compartments are separated by a layer of cells that form a barrier between “inside” and “outside”. This compartmentalized setup mimics the micro-architecture of many tissues and organs, for example microvasculature, renal tubules, and seminiferous cell tubules. Importantly, this setup allows cells to polarize, which is especially important for tissues with barrier functions.

### III. TEM-Chip Design

The TEM-chips used and contemplated in the examples herein are optically clear and constructed in such way that enables compatibility with fluorescent imaging, confocal, brightfield, and phase-contrast microscope imaging. Fluid samples, collected from any of the input or output fluidic ports, can be analyzed using offline techniques such as liquid chromatography, mass spectrometry, ELISA, or gel electrophoresis. In multi-compartment TEM-chips, cell tubes can be perfused independently with media of choice for cell seeding, nutrition and culture maintenance. These media can be supplemented with bioactive agents (for example antibodies, drugs, toxins, or vaccines). For certain studies, the perfusate might be blood, blood components, or blood surrogates. The lumenal fluid path might also serve for administration of microparticles, nanoparticles, single cells, or cell aggregates (for example blood cells, cancer cells, cell spheroids), or microorganism (viruses, bacteria, or parasites). All perfusates can be collected using ports for fluid sampling for further analysis. Additionally, cells can be extracted from the devices to assess gene or protein expression.

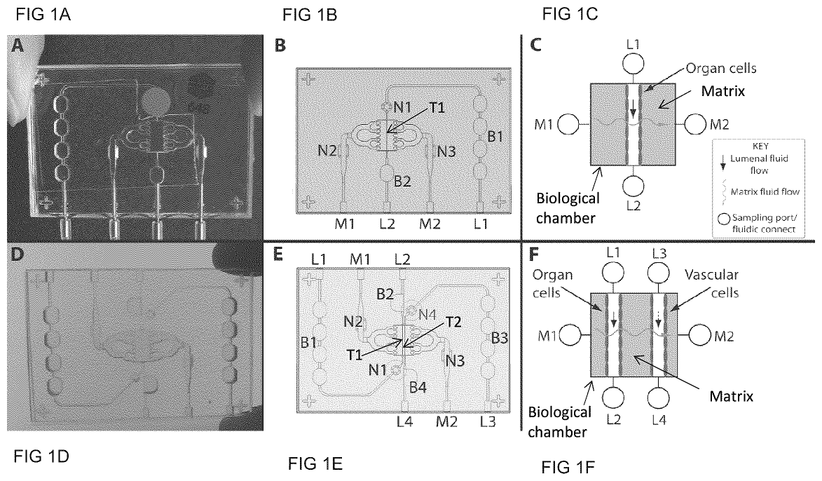

Referring now jointly to FIG. 1A-FIG. 1F, there shown are examples of the two-compartment and three-compartment (single-cell tube and dual-cell tube) TEM-chips, respectively. FIG. 1A-FIG. 1C display a two-compartment chip, and FIG. 1D-FIG. 1F display a three-compartment chip.

Specifically referring now to FIG. 1B and FIG. 1E the technical design of two TEM-chip types is shown where: L1-L2 represents fluidic connections to perfuse the organ cell tube lumenally. L3-L4 represents connections for perfusion of the vascular cell tube. T1 represents the cell tube formed by organ cells; T2 represents the cell tube formed by vascular cells. B1-B4 represent bubble traps. N1-N4 represent areas where a septum can be located, allowing a non-coring septum needle to be inserted for fluid injection or sampling. N1 and N4 also specifically represent the cell injection port, where cells are injected to flow into the void in the biological matrix; thus forming a cell tube or solid cell mass (T1, T2). M1-M2 represent the fluid connections to the extracellular biological matrix, where fluid flow or diffusion of injected compounds takes place. In the devices shown, the preferred method for formation of voids in the biological matrix is using a mandrel, which is inserted into the device at L2 until it reaches N1 (or L4 to N4) prior to injection of the biological matrix via M1 or M2. After the matrix is gelled, this mandrel is removed, leaving a void in the matrix which is fluidically connected to L1-L2 or L3-L4.

Within a single chip as shown in FIG. 1C, there are two separate, independently perfusable compartments: one lumenal compartment, and one matrix compartment. The compartments are separated by the cellular barrier formed by the cell tube. FIG. 1F shows a three-compartment chip schematic, where each of the two cell tubes has separated, independent fluidic connections, in addition to the matrix compartment.

As used in certain applications, multi-compartment TEM-chips can be used to create combinations of structural and/or functional units of organs or tissues in vitro. For example, three-compartment TEM-chips can integrate a tube made from vascular cells together with a tube made from tissue/organ-specific cells. By including a lumenally perfused vascular structure into the system, nutrients can be provided to the tissue/organ-specific cells and metabolic products can be removed, mimicking vascular function in vivo. Other combinations of tubes from various cell sources, with and without blood vessels, are possible.

The matrix compartment mimics the intercellular space in vivo, which plays a significant and complex role on the cellular, tissue, and systemic level. In addition to the cells seeded within the tubular voids, the matrix compartment can be populated with cell types, adding additional flexibility to the design of the microenvironment architecture, for example astrocytes, pericytes, smooth muscle cells, fibroblasts, hepatic cells can be chosen for integration into the extracellular matrix. Many other cell types from various sources, either alone or in combination, are potential candidates to be embedded in the extracellular matrix. Cells can be evenly dispersed throughout the matrix or deposited in specific locations with the matrix. They can be grouped in specific arrangements, combined with other cell types, or embedded as pre-formed structures (such as spheroids). As shown in the preliminary studies using TEM-chips, adding specific cell types to the extracellular matrix compartment influences cellular responses from cells comprising the cell tubes.

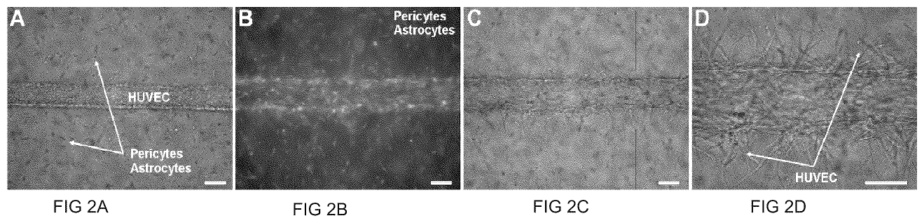

Referring now to FIG. 2A, FIG. 2B, FIG. 2C and FIG. 2D there shown are examples of embedment of cells in a matrix surrounding a vascular-cell tube created of HUVECs. FIG. 2A shows human brain astrocytes and pericytes that are embedded in the proximity of the vascular-cell tube. FIG. 2B shows pericytes and astrocytes that get recruited to the walls of the vascular-cell tube and stimulate HUVEC sprouting (as best shown in FIG. 2C and FIG. 2D). Considering the figures together, it can be seen how cellular responses are affected by the presence of other cell types. Further, non-cellular components (such as micro and nanoparticles, meshes, or slow-release materials) can be added to the matrix as well.

### IV. Examples of Tissue/Organ-Specific Models

The emphasis of the TEM-chip system is to use it with human cells (primary or cultured), in order to study human physiology, pathology, or the response to bioactive compounds such as pharmaceuticals, vaccines, cosmetics or toxic compounds. However, TEM-chips are also applicable to the use of animal cells, such as for the study of animal physiology and pathology, for comparing drug response with data obtained from laboratory animals, and for the study of diseases that are transmitted from animals to humans.

In order to establish a proof of principle, a number of TEM-chip systems were developed. These included:
kidney, intestine, and liver 3D tissue micro-environments as in vitro vascularized organ mimics of “single-organ” functional subunits;
a blood-brain barrier model to demonstrate functionality of a multi-cell barrier type system;
a vascularized tumor model demonstrating the suitability of the assay in tumor biology and for studies on tumor-endothelium interactions; and
an extravasation model for studying the ability of circulating tumor cells to migrate through blood vessels to form metastases.

Systems for studies on example models of functional organ subunits may be built on either two- or three-compartment TEM-chips, with one of the cell tubes representing a blood vessel. In the three-compartment TEM-chips, the distance between the vascular cell tube and the organ-cell tube is kept at <0.5 mm to facilitate diffusion of compounds from the vascular-cell tube to the tissue/organ-like cell tube or vice versa, and for the development of direct cell-to-cell contact between vascular sprouts and organ cells, if that is desired. However, this distance can easily be adjusted as needed.

### Kidney Model

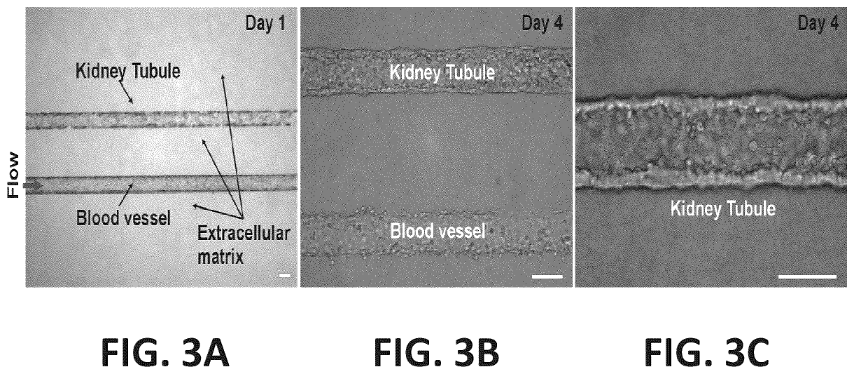

Referring now to FIG. 3A, FIG. 3B and FIG. 3C, an example of a three-compartment model showing a kidney model with a HEK293-tube and a corresponding vascular-cell tube created from HUVECs over a four day period is shown. For the illustrations shown the scale bars=150 μm. For the prediction of renal clearance of drugs and other substances, in vitro models that capture the multicellular complexity and 3D-architecture of the human kidney are highly desirable. The kidney TEM-chip was created by seeding Human Embryonic Kidney cells (HEK-293) into one of the two tubular voids within collagen I. Primary human umbilical vein endothelial cells (HUVECs) were then seeded in the second void and cultured under continuous perfusion with cell culture medium.

Still referring to FIG. 3A, FIG. 3B and FIG. 3C, in the experiment shown the kidney structure was purposely not perfused; exchange of nutrients and metabolic end products was provided solely by the vascular-cell tube. Diffusion of nutrients to and from the vascular-cell tube was sufficient to sustain the culture of kidney cells for at least one week. For functional assessment, lumenal perfusion can be used to examine apical absorption into the cells from the lumen and excretion out of the cells into the lumen, while matrix perfusion can be used to assess basolateral transporter function.

### Intestine Model

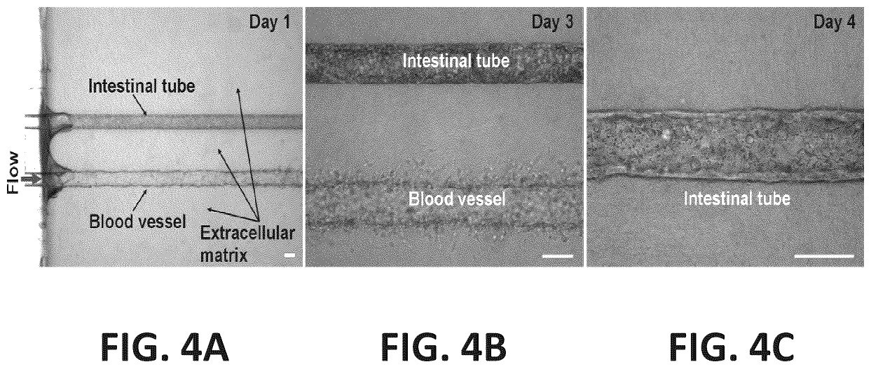

Referring now to FIG. 4A-FIG. 4C, there shown is an example of a three-compartment setup including an intestine-model with a cell tube generated from HT29-cell line and a corresponding vascular-cell tube created from HUVECs. For the illustrations shown the scale bars=150 μm.

Together with the liver, the intestine is involved in first-pass removal of drugs or toxins and is an important barrier tissue that regulates the adsorption of orally administered drugs. The intestinal barrier consists of an epithelial monolayer of cells bound to each other by tight junctions. Substances primarily cross this barrier by membrane diffusion. Predicting the transfer of compounds administered to the digestive tract from intestine to the circulatory system is crucial for the evaluation of drug candidates. However, none of the available in vitro models of the intestinal barrier comprises a vascular component. The intestine TEM-chip include a functional vascular component in parallel with gut epithelium for studies on drug and toxin adsorption.

Referring now more specifically to FIG. 4A and FIG. 4B, human colon carcinoma-derived HT-29 and Caco-2 cells were utilized to form a cell tube generating an intestine-like TEM. Similar to the kidney model, intestinal cells were seeded into one of the tubular voids and allowed to spread. HUVEC cells were seeded into the second void and cultured under constant flow of culture medium. The cell tube with the intestinal cells was not perfused and was maintained by the diffusion of metabolites to and from the vascular-cell tube (as seen also in FIG. 4C).

### Liver Model

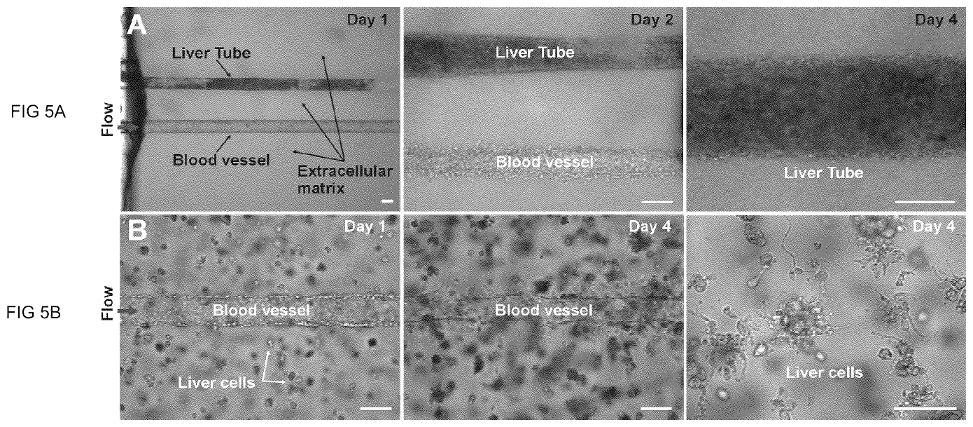

Referring now to FIG. 5A an example of a three-compartment model showing a liver model with a liver-cell tube generated from Hep-G2 cells and a vascular-cell tube generated with HUVECs; both separated by the third compartment is shown. FIG. 5B shows hepatocytes embedded into the matrix surrounding the blood vessel. For the illustrations shown the scale bars=150 μm.

The liver regulates key processes such as blood glucose homeostasis, plasma protein synthesis, detoxification, bile production and transport. Because of the complexity of the liver, in vitro models such as sub-cellular homogenates of the liver, as well as primary hepatocyte cultures that are commonly used to evaluate the biotransformation of drugs, fail to maintain hepatocyte-specific functions in vitro. There is a critical need to develop in vitro models of liver physiology that mimic the 3D microenvironment, including hepatocyte polarity and interactions with other, non-parenchymal liver cells. Furthermore, there is a special interest in systems that allow for consolidation of liver models with other organ models, in particular with a gastrointestinal barrier model and/or a model of the kidney. Together with the liver these organs eliminate drugs and other compounds.

In the liver TEM-chip, human hepatocellular carcinoma cells (Hep-G2). HUVEC cells and collagen-I matrix were used as main components. To mimic the hepatic sinusoid HUVECs were seeded into one of the collagen voids. Hep-G2 cells were seeded into the other of the collagen voids and allowed to proliferate and expand (as shown in FIG. 5A). In an effort to generate structures that resemble hepatocyte plates in vivo, hepatocytes were also embedded into the matrix surrounding the cell tube (as shown in FIG. 5B). The culture was maintained by perfusion of the vascular-cell tube. Such a model can be adapted for the study of the pre-erythrocytic stages of malaria. After initial infection, the malaria parasite travels to the liver where it develops and undergoes a first stage of replication. This stage of parasitic development is of extreme interest to investigators as it represents the most promising target for malaria vaccine development. In the adapted liver chip, the void can be seeded with primary human hepatocytes or established hepatocellular carcinoma cell lines such as HepG2 and HC-04. After seeding, these cells are allowed to proliferate and expand to form cell tubes. In order to generate structures closer to in vivo-like sinusoid liver tissue hepatocytes can also be embedded into the matrix surrounding the cell tube. The cell tube itself can be complemented with other liver sinusoid cells such as Kupffer cells derived from established hepatocyte co-culture cell lines. Parasites can then be injected into the established liver tissue chips, invade hepatocytes to form liver stages, and develop into mature and merozoite-producing liver stages (schizonts) while being maintained by perfusion of the liver cell tube or the surrounding matrix.

In one example using liver cells the microenvironment may be used to culture pre-erythrocytic stages of the malaria parasite Plasmodium falciparum, Plasmodium vivax, Plasmodium berghei, Plasmodium falciparum, Plasmodium ovale curtisi, Plasmodium ovale wallikeri, Plasmodium malariae, Plasmodium knowlesi and/or Plasmodium yoelii.

### Blood-Brain Barrier Model

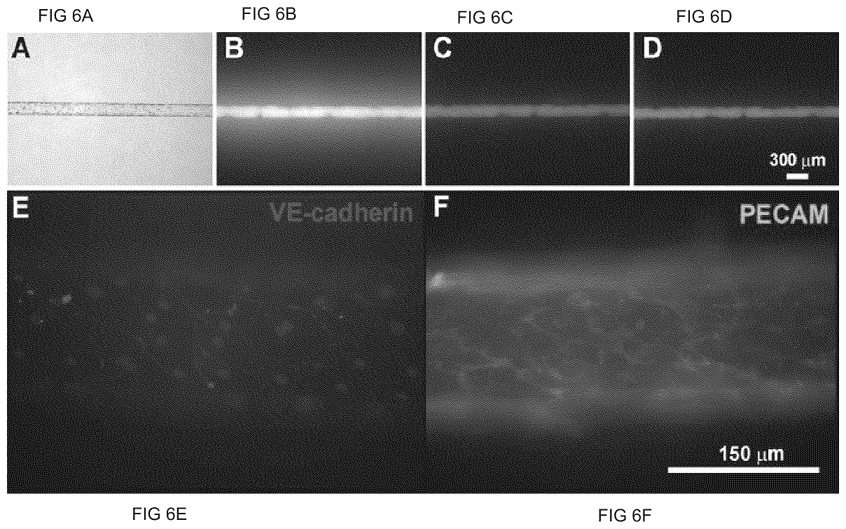

Referring now jointly to FIG. 6A-FIG. 6F show an example of a blood-brain-barrier model in particular illustrating paracellular permeability across the wall of vascular-cell tube (engineered from primary human microvascular endothelial cells) in a two-compartment device.

FIG. 6A shows an oblique illumination microscopic image. FIG. 6B-FIG. 6D are wide-field fluorescence images of a vascular-cell tube after 5 minutes of perfusion. FIG. 6B shows perfusion with Oregon Green, MW 368. FIG. 6C shows perfusion with Alexa Fluor 488-dextran, MW 4 KDa. FIG. 6D shows perfusion with Alexa Fluor 594-dextran, MW 10 KDa. In this example, 14 out of 28 tested vascular-cell tubes were found to be impermeable to BSA, while average permeability through the vascular-cell tube wall was calculated at 1×10⁻⁶ cm/s (N=28), which is comparable to that of isolated mammalian venules (˜2×10⁻⁶ cm/s; Yuan, W. et al, 2009). Permeability of vascular-cell tubes to Oregon Green (2.5×10⁻⁶ cm/s, N=22) is comparable to the values reported for rat brain endothelial cells—astrocytes co-cultures (1.1×10⁻⁵ cm/s; Blasig, I. et al, 2001). Permeability to 10K dextran was found to be similar to in vivo, at 2.7×10⁻⁷ cm/s (N=6). Complete coverage of the vascular-cell tubes wall is demonstrated by the expression of endothelial markers VE-cadherin (as shown in FIG. 6E) and PECAM (as shown in FIG. 6F).

The blood-brain barrier model is designed as a two-compartment TEM-chip. A blood-brain barrier model was created from the cell types that comprise the human brain neurovascular unit including microvascular endothelial cells, pericytes and astrocytes. This tissue-like environment contains human brain pericytes and astrocytes embedded in 3D extracellular matrix (ECM) that support the vascular-cell tube, thus mimicking the in vivo architecture and allowing physical contact between the different cell types. This vascular-cell tube is exposed to lumenal flow. Test drugs can be added to the fluid path that runs through the vessel. Drug penetration through the vessel can be measured by analyzing the fluid collected outside the vessel (ECM washout), or by visualizing the drug with fluorescent tracers.

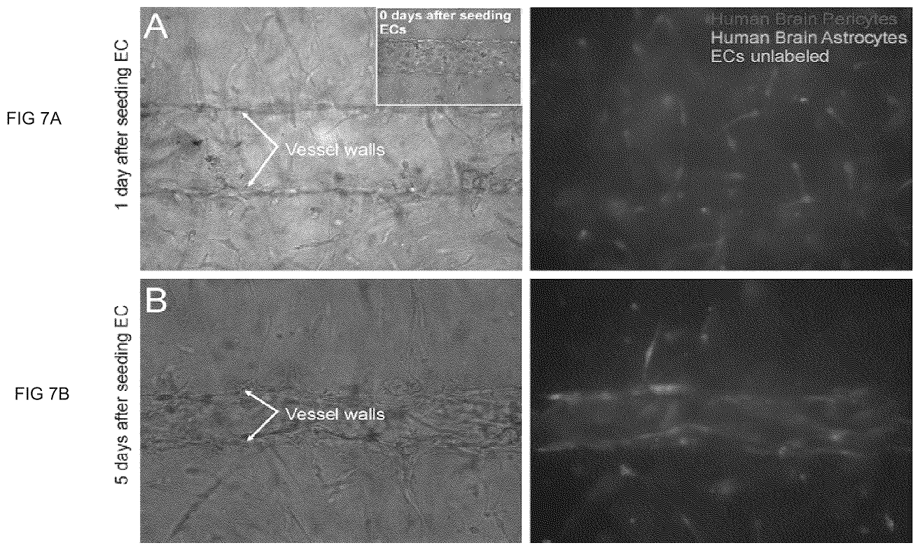

Referring now to FIG. 7A and FIG. 78, a re-organization of cells in the BBB model consisting of hCMEC/D3 (human brain microvascular cell line) and ECM-embedded pericytes and astrocytes (primary human brain cells) is shown. Specifically FIG. 7A shows astrocytes and pericytes, embedded in the matrix leads to close association of these cell types with the ECs, causing a gradual decrease in vessel diameter as seen in FIG. 7B.

The results demonstrate that the vascular-cell tubes display morphological and functional characteristics of microvascular endothelium in vivo. The cells within the vascular-cell tubes possess endothelial morphology and show typical pericellular localization of endothelial markers (as best shown in FIG. 6A-FIG. 6F). Cells form a tightly packed layer with contact-inhibited morphology. Both, matrix-embedded astrocytes and pericytes are recruited to the vascular-cell tubes and exert a profound influence on its morphology (as best shown in FIG. 7A and FIG. 7B). The barrier functions obtained with the BBB model are similar or superior to published data on other in vitro BBB models.

### Cancer Model

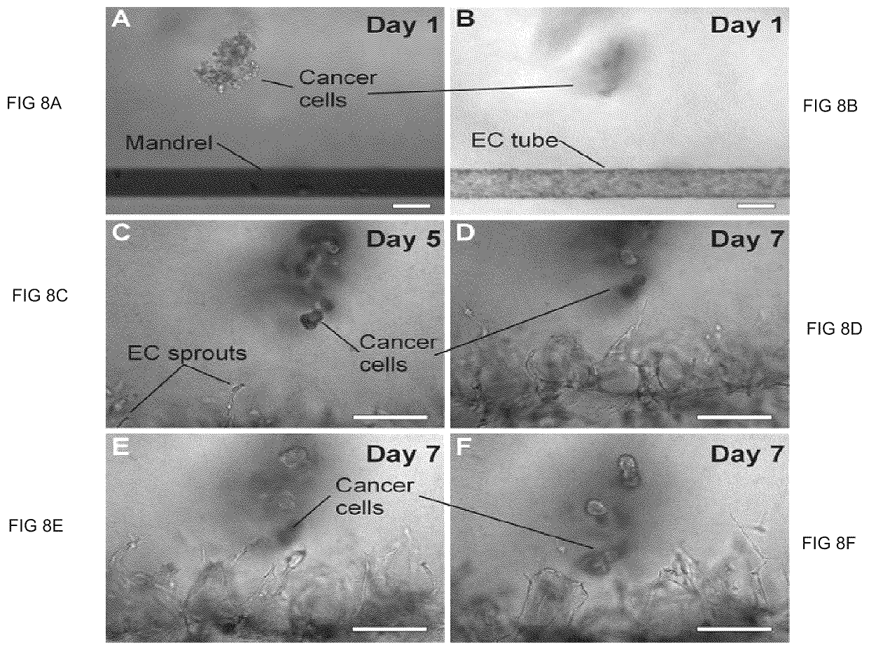

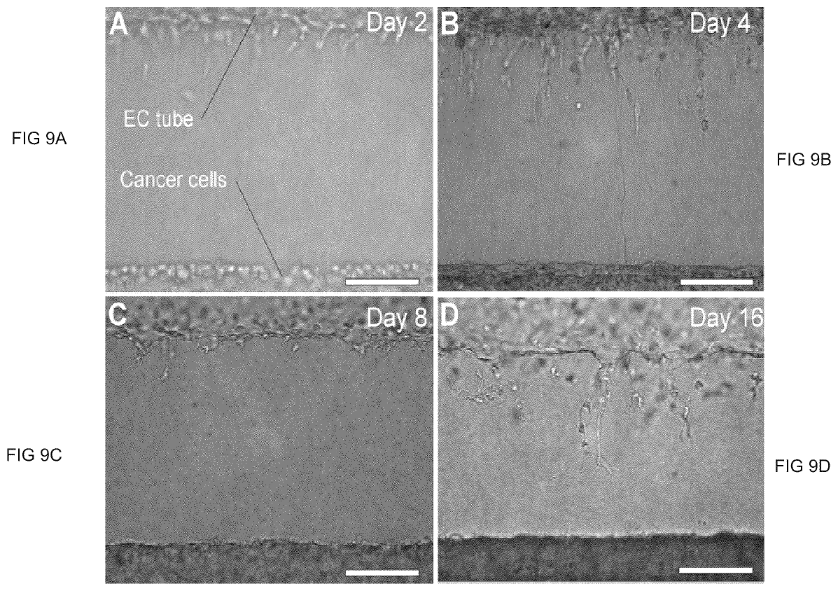

The cancer TEM-chip was developed to allow for studies on interactions of cancer cells and cells of the microvascular endothelium, such as homing signals during intra- and extravasation, tumor angiogenesis, and markers expressed by the neo-vasculature. Importantly, the model allows for the screening of anti-cancer drugs and evaluation other therapies, such as the effect of radiation on cancer cells and on tumor vasculature. In the three-compartment chips one of the cell tubes can be populated with cancer cells in the form of a cell tube or cell cylinder (as shown above with reference to FIG. 3A, FIG. 3B and FIG. 3C) while the other cell tube can be seeded with endothelial cells in order to generate a vascular-cell tube with the ability to sprout toward the cancer-cell tube (see FIG. 8A-FIG. 8F and FIG. 9A-FIG. 9D discussed in more detail below).

Referring now to FIG. 8A-FIG. 8F, an in vitro image of an example of a two-compartment model of tumor-endothelium interactions over a period of 7 days. Specifically referring to FIG. 8A, there shown are cancer cell cluster of BT-474 cells (breast cancer cell line) that were embedded in collagen in the proximity of a mandrel that is used to create a tubular void. HUVECs were then seeded into the void as shown in FIG. 8B. FIG. 8C-FIG. 8F are close-up views of the sprouts that grew from a “parent” HUVEC-tube toward the cancer cells. For the illustrations shown the scale bars=150 μm.

Referring now to FIG. 9A-FIG. 9D jointly show an example of a three-compartment model of tumor-endothelium interactions over a 16 day period. FIG. 9A shows Caco-2 (human colorectal adenocarcinoma) cells that were deposited in one collagen void (bottom tube) after the HUVEC tube was formed (top tube). FIG. 9B shows sprouts that formed from a parent HUVEC vessel four days after seeding. FIG. 9C and FIG. 9D show human liver carcinoma cells (Hep-G2 cell line) that were deposited in the bottom collagen channel and HUVECs were seeded in the top channel. For the illustrations shown the scale bars=150 μm.

For the experiments human breast cancer cells (BT-474), colorectal adenocarcinoma cells (Caco-2), and hepatocarcinoma cells (Hep-G2) were used. Cancer cells were seeded into one of two tubular voids and HUVECs in the other. The cultures were maintained by perfusion through the vascular-cell tube (HUVEC tube) only; the cell tube populated with cancer cells was not perfused. As shown, for example, in FIG. 8C and FIG. 9D, the vascular-cell tubes developed sprouts that were directed toward the cancer-cell structures.

### Cancer-Cell Extravasation Model

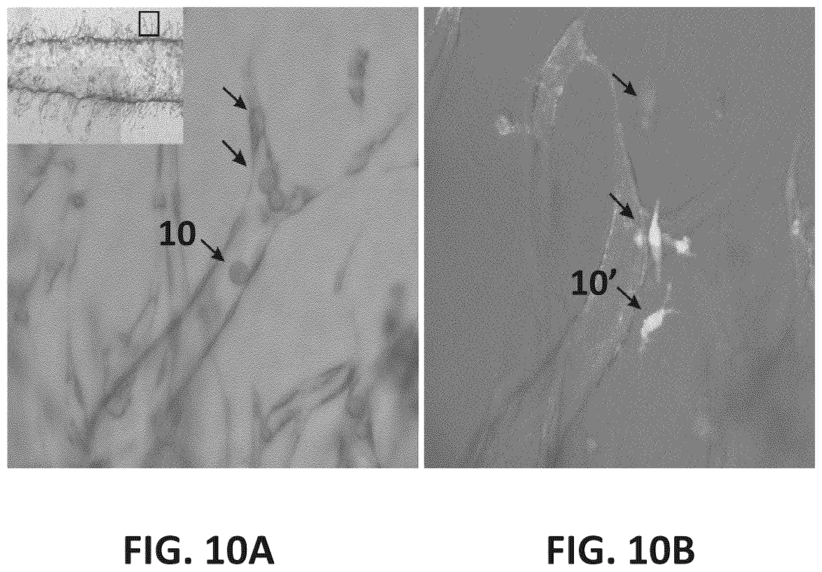

Referring now jointly to FIG. 10A and FIG. 10B, an example of a cancer cell extravasation is shown. Specifically referring to FIG. 10A, fluorescently-tagged prostate cancer (PC3) cells are lumenally administered to a HUVEC tube where they adhere to the inner wall of the endothelial sprouts as indicated by arrows 10.

Now referring to FIG. 108, the progression of extravasation can be monitored continuously. 20 hours after seeding, PC3 cells have migrated through the endothelium into the surrounding ECM right image as indicated by arrows 10′.

Extravasation is the ability of circulating tumor cells to migrate through blood vessels to form metastases. The mechanisms by which tumor cells penetrate the endothelial cell junctions remain one of the least understood in cancer progression, in part due to the lack for appropriate models. The study of factors that influence mechanisms by which tumor cells penetrate endothelial cell layers is expected to translate into new cancer therapeutics. Only one type of in vitro model is currently commercially available for the study of extravasation: the Boyden-Chamber/Transwell-Invasion-Assay, developed for studies on chemotaxis by Boyden in the 1960s. While inexpensive and easy to perform, this assay does not allow real-time observations of tumor cells and endothelium. In addition, this assay addresses tumor cell migration under static conditions, despite the important role of shear stress on interactions between endothelium and circulating tumor cells as well as tumor cell deformation. The TEM-chips allow for the real-time study of tumor-cell extravasation using sprouting microvasculature within a tissue-like matrix in the presence of lumenal flow. Furthermore, the model allows to add and to vary key elements, such as additional cells, extracellular matrix, growth factors, as well as perfusion parameters and other physical conditions. For example, the matrix can be populated with different stroma cells (normal, reactive, or senescent), various cancer-cell types, or patient-specific cells (for personalized drug testing).

The cancer-cell extravasation TEM-chip was designed using both the two- and the three-compartment setups. In the two-compartment devices, single “parent” vascular-cell tubes are created, which are subsequently induced to angiogenic sprouting. To test their extravasation potential in the system, suspensions of fluorescently-tagged (i.e. with CellTracker dyes) highly metastatic PC-3 prostate carcinoma cells (as best seen in FIG. 10A) were added to the lumenal fluid flow and deposited into the vessel sprouts. The extravasation potential is measured by determining the fraction of cancer cells that have migrated through the endothelial sprouts into the matrix within a certain time frame versus the fraction of cancer cells that remain trapped within the sprouts.

In the three-compartment devices two “parent” vascular-cell tubes are created whose sprouts subsequently anastomose and form capillary networks. Fluid flow can be routed from one “parent” cell tube via the capillary network into the second “parent” cell tube—resembling a vascular bed with an arterial and a venous end. Cancer cells can be circulated through this vascular bed for evaluating their metastatic potential. Their progression through the endothelial tubule wall can then be monitored continuously or in time intervals.

### Integration of Different Tissue/Organ Models

The TEM-chip design allows using individual chips as single modules that can be integrated with others into a larger platform, thereby creating multi-organs setups that have physiological and pathological significance, such as a combination of intestine, liver and kidney modules. Platforms with two, three and up to 10 TEM-chips, each representing the same or different tissue/organ types are proposed for development.

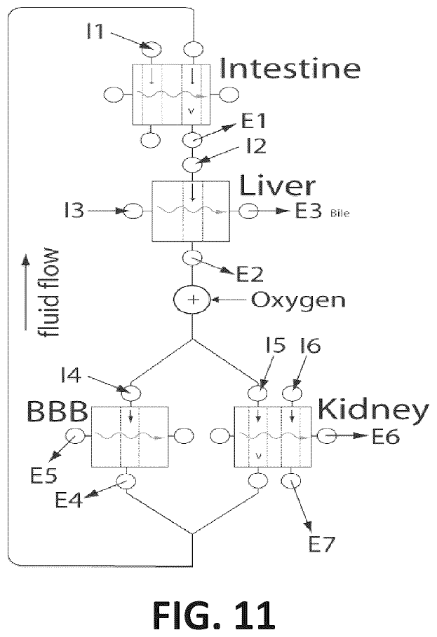

These integrated multi-organ platforms will present novel ways to investigate toxicological effects of drug candidates and other substances, not only on individual organ cultures but also on complex organ systems of multiple organ models in corresponding sequences (e.g. intestine, liver and kidney). Such setups can include combinations of structural/functional subunits of the same organ (e.g. proximal with distal kidney tubule) or different organs (e.g. intestinal barrier with liver and blood-brain-barrier). Circulatory and unidirectional flow systems have been designed. FIG. 11-FIG. 13 demonstrate fluidic setups integrating four to 10 TEM-chips as discussed hereinbelow.

Referring now to FIG. 11, an example of four connected TEM-chips forming a complex system with each chip representing a different organ is illustrated. A central, two-compartment liver TEM-chip, connected to kidney, intestine and BBB TEM-chips. Other organ-type TEMs can be added as desired by the investigator. All modules share a common fluidic path which represents vascular (“blood”) flow. Oxygen may be diffused into the flow to take the place of physiological systems not present, such as the lung. 11 represents a port for injection of nutrients to be absorbed by the intestine cell tube and passed to the vascular cell tube. E1 represents a port for extraction of fluid for analysis, such as glucose monitoring. Note that sensors can be directly inserted at these points for measurement (examples: oxygen, pH). 12 represents a port for injection of compounds to be buffered/absorbed by the liver, E2 represents a port for extraction of the fluid filtered by the liver-chip; studying the change in concentration of said compound and its kinetics indicates a preliminary liver functionality. 13-E3 represent ports for extraction of bile from the liver module. 14 represents a port for injection of a compound for blood-brain barrier testing, where ports E4 and E5 are sampled for measurement of barrier function. 15 represents the port for injection, for example, of nitrogenous substances into the kidney-chip. Port E7 is sampled from the proximal tubule of this kidney module, and analyzed for nitrogenous substances. Other kidney function can be demonstrated by injection of glucose solution at port 16, leaving port E6 open to atmosphere and checking if glucose solution collects in the matrix compartment.

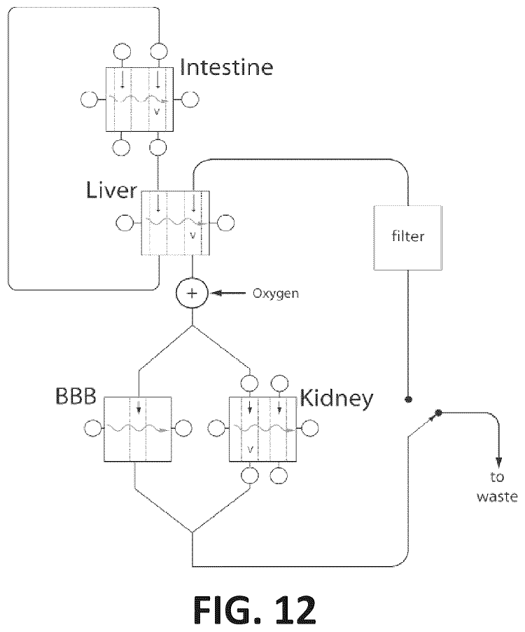

Referring now to FIG. 12, an example of an alternative architecture for connecting four TEM-chips, each representing a different organ, to a complex system is illustrated. A central, three-compartment liver-chip, and is connected to the kidney, intestine and BBB TEM-chips.

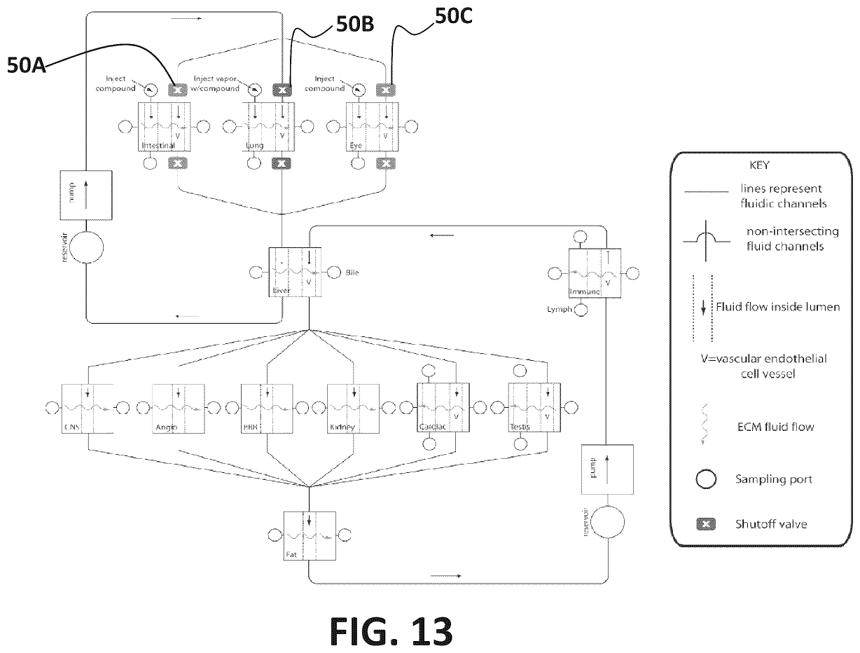

Referring now to FIG. 13, an example of an alternative architecture illustrates an example of an alternative architecture employing a plurality of many more physiological modules integrated into one circuit. One of three shutoff valve pairs 50A, 50B and 50C is active at any given time. A (not-shown) recirculating pump for shutoff sections may be required.

Up to this point a microfluidic system for generating multiple compartmentalized microenvironments of tissues and organs in vitro has been described. The system disclosed above allows independent perfusion of the separate compartments. The system is designed for generating in vitro models of tissues and organs that mimic in vivo functionality.

To briefly recap, a microfluidic device contains a chamber that has been filled with a matrix that surrounds at least one void. The fluidic channels of the device are connected to the chamber in such a way that fluid flowing through the void has no connection with the matrix and fluid flowing through the matrix has no connection with the void. Multiple cell types can be seeded into the void, where the cells can form functional tissue or organ units. The cells within the void are separated from the matrix by a cellular membrane that forms a barrier. Thus, no artificial materials are required for cell attachment or scaffolding. Key features of this system include: the compartmentalized setup, lack of artificial materials, and ability for independent perfusion.

Together, these features allow the system to closely mimic the in vivo environment and allow the user the flexibility to study multiple aspects of tissue biology. In particular, the ability to independently perfuse compartments separated by a cell membrane allows one to carry out previously unfeasible experiments. These include experiments related to cell barriers such as investigating the barrier capabilities of specific cells in response to different stimuli and investigating the transport of different compounds across a cellular barrier. Further, investigators can independently sample multiple compartments to isolate different cellular outputs, like cytokines or drug metabolites. Gradients can be created from one void, and cellular impact studied in separate tissues or cells populating a second void. Finally, the system allows the user to study interactions between multiple tissues. This is particularly important when connecting multiple microfluidic devices to understand how different tissues and stimuli interact. Using the system, the modules will share a common fluidic path that represents vascular (“blood”) flow, allowing investigators the ability to accurately predict how compounds will be metabolized and tissue function impacted in response to a variety of stimuli.

### Description of Applications for Reproducing Functional Units of Invertebrate Tissues and Organs as Culturing Environments for Parasites

Having described the basic microfluidic devices hereinabove, more specific applications for these devices will now be addressed, specifically with respect to vaccine research applications. While examples herein address mosquito midgut chips and cells, the invention is not so limited. It will be understood by those skilled in the art having the benefit of this disclosure that cells of other invertebrates may be employed for various other applications. For example, it is contemplated that tick cells may be used in a tick cell chip for purposes of analyzing potential drugs related to tick borne diseases such as, for example, Lyme disease and other related conditions. Similarly, cells from fruit flies may be employed to make testing chips for parasitic diseases, including malaria and others. While the examples herein address the recapitulation of a mosquito midgut environment, it can be used also for generating other invertebrate tissues, for example a mosquito salivary gland microenvironment for the culture of Plasmodium sporozoites.

Referring again concurrently to FIG. 1A-FIG. 1C, there shown is an example of a TEM-Chip design with FIG. 1A showing a photograph of a readily assembled TEM-Chip and FIG. 1B displaying a schematic of the two-compartment system in detail as built by Nortis, Inc. of Seattle, Wash. Within a single chip as shown in FIG. 10, there are two separate, independently perfusable compartments, one lumenal compartment, and one matrix compartment. The compartments are separated by the cellular barrier formed by the cell tube. In the Nortis chips, the matrix compartment comprises the extracellular matrix, which naturally surrounds tissues and blood vessels in the form of connective tissue or interstitium. Together, both compartments result in the unique architecture of the culturing device, and yield a substantial benefit that is novel and unique to the system: if necessary, the cell tube constituting the midgut tissue can be provided with nutrition through media flow from the side perfusion ports and around the cell tube instead of being applied through the engineered cell tube. This spatial separation of flow from the tissue-specific cells and the cell tube lumen protects both from damage and disturbance due to shear stress from medium flow while it at the same time allows optimal nutritional support by diffusion.

In addition to the cells seeded within the tubular voids, the extracellular matrix compartment can be populated with cells as desired for individual experimental designs. This compartment can be perfused independently from the cell tube and samples can be taken for cellular or biochemical analysis. Primary mosquito midgut cells or cells from established mosquito cell lines can be chosen to be integrated and embedded into the extracellular matrix. Other cell types from various sources (e.g. D. melanogaster), either alone or in combination, are potential candidates to be embedded in the extracellular matrix as well.

The option for populating the matrix-compartment with additional cells and cell types allows for additional variables of the experimental conditions, e.g. stimulation of conditioning of the cell culturing medium and environment, resulting in manipulation of cell proliferation, growth and organization. As shown in preliminary studies with other tissue microenvironments, adding specific cell types to the extracellular matrix compartment (typically done by mixing cells into the biological matrix) influences cellular responses from cells comprising the cell tubes.

### Example I

### Mosquito Midgut Microenvironment

The emphasis of the herein-disclosed TEM-Chip system is for use with mosquito midgut cells (primary or cultured) in order to generate a mosquito midgut-like physiology and to create a microenvironment that allows for the successful culture of Plasmodium falciparum insect stages. However, the TEM-Chip described here can be used to culture other Plasmodium species as well, such as Plasmodium vivax or the murine parasite species of Plasmodium berghei, Plasmodium falciparum, and Plasmodium yoelii. This system will provide an optimized platform for testing of potential malaria vaccine candidates, transmission blocking vaccine candidates or other antimalarial compounds and allow a substantial improvement on in vitro malaria parasite cultures and of the current “gold standard” of classic membrane feeding assays.

In order to establish a proof of principle for the suitability of the TEM-Chips as a culture environment for mosquito cells, a preliminary system was developed in which cells were seeded from an established mosquito cell line into the TEM-Chip and cultured to confluence. This system is built on a two-compartment chip with the cell tube representing a mosquito midgut-like structure.

The extracellular matrix compartment was composed of Collagen I and the inner, surface of the void was coated with poly-L Lysine prior to cell seeding. The coating of the Collagen I surface was accomplished by lumenal perfusion of the collagen void with a 10 μg/ml poly-L Lysine solution for 1 hour at room temperature and at a flow rate of 0.25-5 μl/min.

The cell tube was formed by cultured, immortalized mosquito cells (4A-3B cells) which were derived from a cell preparation of mosquito larvae and published and deposited to ATCC/MR4 previously (George K. Christophodes, Imperial College. London, 2002). The cells were harvested from cell culture vessels after mild trypsinization and injected through the N1 septum at a concentration of ˜1 million cells per ml and circulated for about 15 minutes at room temperature and at a flow rate of 5 ml/min. The culture of 4A-3B cells was maintained with Schneiders insect culture medium supplemented with 10% inactivated Fetal Bovine Serum in both, the original culture vessel and inside the TEM-Chip. After seeding, the flow rate for fresh medium was maintained overnight and cells were left to adhere to the lumen walls of the collagen void. Subsequently, the cells were cultured within the chip for up to 5 additional days with constant flow of fresh medium at 0.25-5 μl/min to ensure viability, lasting cell adhesion and cell maintenance within the chip.

As a result, the tissue generated from those mosquito cells forms as a circular, single-cell monolayer coating the internal surface of the cell tube lumen, thus forming the desired cell tube just as anticipated. The cells were provided with nutrition by perfusion through the cell tube lumen.

With preliminary experiments using immortalized embryonic mosquito cells already showing great promise, it is believed that other mosquito-derived cell types will be equally functional in the chips. Thus, using primary or established cells directly isolated from freshly dissected mosquito midguts is planned for use for future experiments to ensure closest similarity of the engineered mosquito midgut environment to the native tissue.

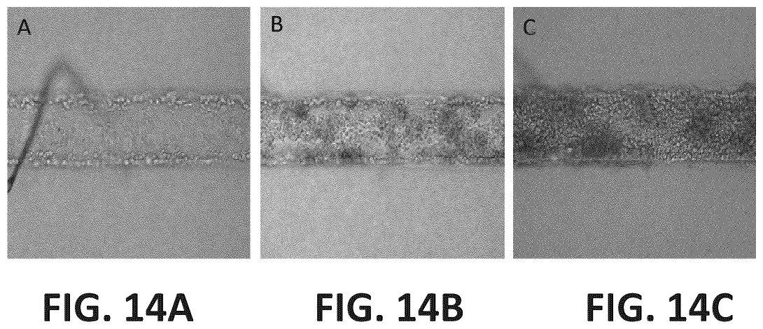

Referring now concurrently to FIG. 14A-FIG. 14C, an example of a two-compartment mosquito midgut chip showing a cell tube with a mosquito 4A-3B-cell coated tubule developing over a time period of 5 days is shown. The images show the mosquito cell chip seeded with mosquito cells and the formed mosquito cell tube within the TEM-Chip. FIG. 14A shows a collagen void with seeded cells on day 1. FIG. 14B shows the same cell tube of FIG. 14A two (2) days later with mosquito cells attached and spreading. FIG. 14C shows the same cell tube on day 5 with cells still attached and grown to confluence.

### Example II

Plasmodium falciparum Culture Environment

In one useful embodiment a chip-model for the creation of a culture environment for Plasmodium insect stages within the mosquito midgut chip described above was designed. The targeted end point stages are the sporozoite-producing oocysts, a late stage in the parasite life cycle which requires the completion of a number of viable earlier stages and thus needs to take place within an optimal culture environment. Plasmodium parasites undergo repeated replication in an asexual life cycle that occurs in red blood cells (RBCs) within the host blood stream.

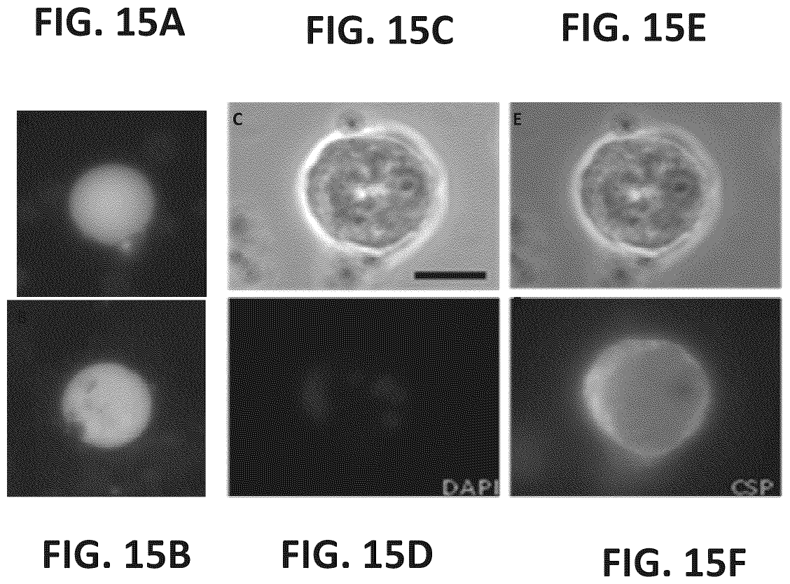

Over time, some of the developing parasites develop into sexual stages and rest, instead of developing into further replicating asexual stages. Once transferred into a mosquito midgut after a blood meal, the mature sexual stages (gametocytes) leave the RBCs, fertilize each other and transform into motile ookinetes. These cells actively leave the midgut environment by passage through the midgut epithelium and settle at the outer interface between epithelium and surrounding basal lamina, which in turn is surrounded by the mosquito hemolymph. There, the ookinetes will transform into oocysts and begin to grow, then produce and eventually release the infectious stages of sporozoites which then are transferred by the mosquito to the next host, perpetuating the cycle. Thus, in order to create a culture environment that supports the development of oocysts, an environment must be provided that allows for fertilization and ookinete formation. Previous research that has been adapted and optimized by another laboratory during previous projects is available and has led to the identification of those conditions and allows us to produce early stage oocysts in very preliminary culture environments (see FIG. 15A-FIG. 15F described below).

Referring now concurrently to FIG. 15A-FIG. 15F examples of early stage oocysts in very preliminary culture environments are shown.

Referring now specifically to FIG. 15A there shown is an example image of a GFP expressing oocyst generated with the setup shown in FIG. 1.

Referring now specifically to FIG. 15B there shown is an example image of the in-vitro generated oocysts, showing that they are identical in size and shape to oocysts generated in vivo with the membrane feeding assay.

Referring now specifically to FIG. 15C-FIG. 15F there shown is an example of in-vitro generated oocysts expressing circumsporozoite protein (CSP), which is an indicator for proper development. (FIG. 15C) phase contrast, (FIG. 15D) DAPI, (FIG. 15F) CSP label, (FIG. 15E) overlay; where the scaling bar=10 microns as shown in FIG. 15C.

To promote further understanding of the method and system of the invention, hereinbelow a far more sophisticated approach by applying previously determined culture conditions to the of mosquito midgut chips is disclosed for the first time.

Preliminary research has indicated a beneficial effect of mosquito cells co-cultured with the parasites and the use of culture medium enriched by several factors, amongst them extracts from red blood cells and mosquito pupae. By using the Nortis TEM chips substantially all of those needs may be accommodated and a culture environment can be provided that is furthermore optimized by another, critical feature. In contrast to all other approaches previously published, the co-cultured cells in the mosquito midgut TEM-Chips have the ability to polarize their cell architecture to an “inside” and an “outside” cell surface assembly, therefore offering a repertoire of cell surface receptors to the migrating ookinetes that is much closer to the native environment than achieved in any previous work.

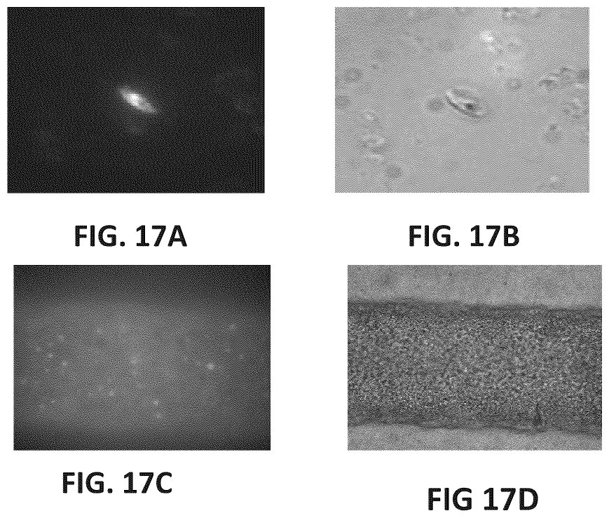

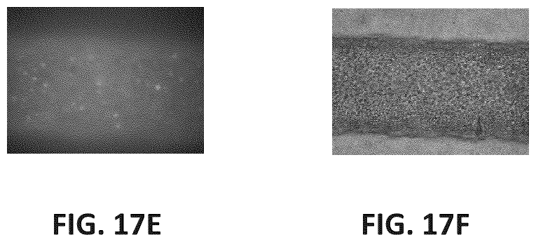

To be able to easily visualize cultured parasites transfected parasites will be used that constitutively express luciferase and green fluorescent protein (GFP) and which were produced previously in a different laboratory. A suspension of red blood cells is injected with an enriched, high concentration of mature parasite gametocytes or enriched parasite ookinetes (See FIG. 17A-17F) into the lumen of the mosquito midgut chip until a state of densely packed RBCs completely filling the lumen of the generated midgut is reached. The gametocytes will be produced using previously developed protocols. 16-day cultures of sexually determined parasites will be cultured to maturity at 37. degree. C. Subsequently and prior to injection, the parasites will be enriched to allow for higher exflagellation rates, fertilization efficiency, and ookinete yields. Enrichment will be achieved by concentrating the parasites magnetically over MACS columns (Miltenyi). This convenient approach is possible due to the parasites' content of iron-hemozoin. This technique yields a ratio of up to 50% of parasite-containing red blood cells. A drop in temperature to 26. degree. C. will induce parasite exflagellation which will result in fertilization and ookinete formation within the RBC-packed midgut lumen when the conditions are optimal.

During the following first 24 hours post-inoculation the culture will be maintained by perfusion with an “ookinete medium” developed based on published protocols. Perfusion with culture medium will be maintained either through the cell tube or through the side perfusion ports. After 24 hours at 24-26° C., ookinetes should be fully developed, motile and leaving the midgut lumen; a process that we anticipate to be able to monitor microscopically and in real time without interrupting the culture due to the clear material of the TEM-Chip.

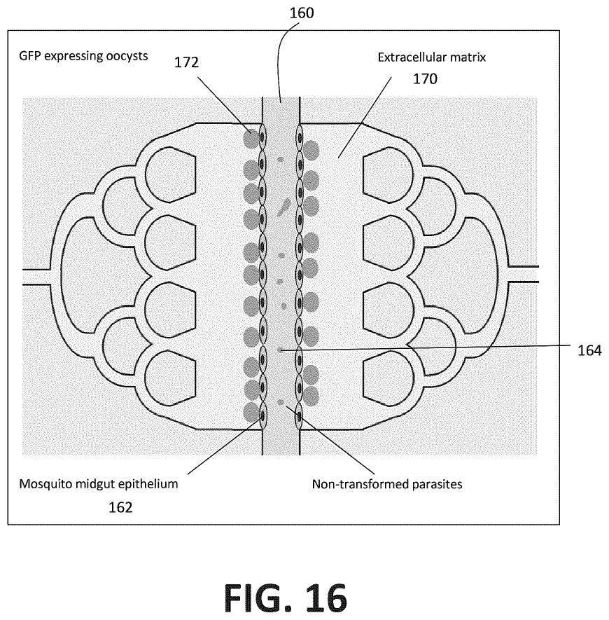

After completion of ookinete development, the medium will be replaced by “oocyst medium”, previously developed by the group, and perfused through the side ports and the tube-surrounding matrix. As before, during this process the developing culture within the cell tube will be continuously monitored. Once sessile on the ablumenal side of the midgut wall the parasites are anticipated to transform into oocysts; however, culture conditions and co-culture with mosquito cells might have to be adjusted to achieve optimal results, e.g. by seeding additional mosquito cells inside the tube-surrounding matrix to further condition the culturing medium. After 10⁻¹² days of continuous culture, the number of developing oocysts per mosquito midgut chip can be counted manually or in an automated manner since the GFP-expressing parasites will be emitting strong fluorescence that can easily be detected by automated microscope and camera software. For a schematic image displaying a projection of how the system is expected to develop at day 12 see FIG. 16 described below.

Referring now specifically to FIG. 16 there schematically shown is an example of a midgut chip. The lumen 160 of a cell tube created from mosquito midgut epithelial cells 162 is loaded with a suspension of malaria-infected red blood cells. The parasites 164 undergo sexual reproduction and migrate through the midgut epithelium into the surrounding matrix 170 where they transform into oocysts (OC) 172. OCs appear as brightly fluorescent spheres (.about.20 micron diameter). Assay readout is the number of OCs on the ablumenal side of the midgut: the smaller the OC count the higher the transmission-blocking activity of the test compound. The microenvironment is maintained by perfusion with growth medium.

Once feasibility is robustly established, the midgut chip and culture conditions may be optimized to increase the yields of oocysts per midgut microenvironment and thus to increase statistical relevance of experiments possible per chip. Besides optimizing the culture conditions, the characteristics of the Nortis TEM chip and manner of its fabrication allows creating longer midgut tubes or arrays of multiple midgut tubes within one chip. This can increase the overall culture volume and surface for oocysts to settle and develop. Thus, several hundred oocysts per chip could be achievable.

With an oocyst culture protocol established the system can be applied to studies on potential compounds for malaria vaccines or transmission blocking vaccines. However, with oocysts developing to a state of maturity, the system will provide the first option ever described to produce large numbers of Plasmodium falciparum sporozoites in vitro—a vital step to produce a much needed malaria vaccine.

Referring now to FIGS. 17A-17D, an example of enriched GFP-expressing Plasmodium falciparum ookinete 48 hrs post-fertilization in suspension of RBC's is shown. FIG. 17A and FIG. 17C were taken under GFP fluorescence. FIG. 17B and FIG. 17D were taken using transmitted light.

Referring now to FIGS. 17E-17F, there shown are examples including a cell tube of 4A-3B cells with injected GFP-expressing parasites in stages of zygotes and developing and matured ookinetes. FIG. 17E was taken under GFP fluorescence. FIG. 17F was taken using transmitted light.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices and reconstruction algorithms, and that various modifications, both as to the equipment details and operating procedures, may be accomplished without departing from the true spirit and scope of the present invention.

What is claimed is:

1. A microfluidic system for reproducing functional units of tissues and organs in vitro comprising: a plurality of microfluidic devices having at least a first perfusion path and a second separate perfusion path; the plurality of microfluidic devices each also having a chamber containing a matrix, where the matrix surrounds at least one void whose lumen is in fluidic connection exclusively with the first perfusion path, where the at least one void is populated with at least one cell type in such way that the cells are in direct contact with the matrix; where the matrix is in fluidic connection exclusively with the second separate perfusion path; wherein the plurality of microfluidic devices are integrated onto a platform; and wherein each of the plurality of microfluidic devices mimics at least a partial organ module.

2. The system of claim 1 wherein the organ modules are selected from the group consisting of intestine, liver, kidney, and blood-brain barrier modules.

3. The system of claim 1 wherein the plurality of microfluidic devices are connected to form a complex system with each microfluidic device representing a different organ type.

4. The system of claim 2 wherein a central, two-compartment liver module is connected to a kidney module, an intestine module and at least one BBB module.

5. The system of claim 1 where organ modules share a common fluidic path, which represents vascular flow.

6. The system of claim 2 further comprising: an inlet coupled to at least one path for oxygen diffusion; a port coupled to at least one path for injection of nutrients to be absorbed by an intestine cell tube and passed to a vascular cell tube; a port coupled to at least one path for extraction of fluid for analysis; a port coupled to at least one path tor injection of compounds to be buffered/absorbed by the a liver module: a port coupled to at least one path for extraction of the fluid filtered by the liver module; at least one port coupled to at least one path for extraction of bile from the liver module; a port coupled to at least one path for injection of a compound for blood-brain barrier testing; a port coupled to at least one path for injection of nitrogenous substances into the kidney module; and a port coupled to a void for injection of cells.

6. The system of claim 2 further comprising a plurality of shutoff valves located to control fluid flow through selected organ modules.

7. A system for reproducing a functional unit of an invertebrate tissue in vitro, as a tissue-engineered microenvironment comprising: a microfluidic device having at least a first perfusion path and a second separate perfusion path; the microfluidic device also having a chamber containing a matrix, where the matrix surrounds at least one void whose lumen is in fluidic connection exclusively with the first perfusion path, where the at least one void can be populated with at least one invertebrate cell type in such way that the cells are in direct contact with the matrix; and where the matrix is in fluidic connection exclusively with the second separate perfusion path.

8. The system of claim 7 wherein the invertebrate tissue-engineered microenvironment is utilized for the culture of parasites.

9. The system of claim 7 wherein the invertebrate cells are selected from the group consisting of mosquito midgut cells, mosquito cells, primary invertebrate cells, cultured invertebrate cells, primary mosquito midgut cells, cultured mosquito midgut cells, fly cells, bug cells, tick cells and fruit fly cells.

10. The method of claim 8 wherein the invertebrate cells comprise mosquito midgut cells and culturing parasites includes culturing the malaria parasite Plasmodium falciparum insect stages, Plasmodium vivax or the murine parasite species of Plasmodium berghei, Plasmodium falciparum, and Plasmodium yoelii.

11. The method of claim 7 wherein the void is populated with mosquito midgut cells to generate a mosquito midgut structure with in-vivo like organ physiology.

12. The method of claim 8 further comprising using the testing microenvironment for testing of potential malaria vaccine candidates, transmission blocking vaccine candidates or other antimalarial compounds.

13. The system of claim 1 wherein the cells populating the at least one void are liver cells and the microenvironment is used to culture pre-erythrocytic stages of the malaria parasite Plasmodium falciparum, Plasmodium vivax, Plasmodium berghei, Plasmodium falciparum, Plasmodium ovale curtisi Plasmodium ovale waffikeri, Plasmodium malariae, Plasmodium knowlesi and/or Plasmodium yoelli.

14. The system of claim 3 wherein the microenvironment is used for testing of potential malaria vaccine candidates, transmission blocking vaccine candidates, or antimalarial compounds.

15. The system of claim 7 wherein the voids are seeded with cells from mosquito salivary glands.

16. The system of claim 7 wherein the at least one void is seeded with cells from an established insect cell line.

17. The method of claim 7 wherein the at least one void is perfused and thus coated with amino acids or proteins that enhance cell adhesion.

18. The system of claim 7 wherein the void is populated with immortalized mosquito cells derived from a cell preparation of mosquito larvae and published and deposited to ATCC/MR4 previously.

19. The method of claim 18 wherein the cells comprise 4A-3B cells.

20. The method of claim 7 wherein the at least one void is perfused with materials selected from the group consisting of nutrient solutions, test substances, blood, blood components, and blood surrogates.

21. The method of claim 7 wherein the microfluidic device is fabricated from a polymer selected from the group consisting of a polymeric organosilicon compound, silicone, polydimethylsiloxane (PDMS), cyclic olefin copolymer, polystyrene, and polycarbonate.

22. The method of claim 7 wherein the chamber and paths are embedded in a substrate juxtaposed between a glass plate and a polycarbonate, or rigid clear thermoplastic, plate.

23. The method of claim 7 wherein the matrix is selected from the group consisting of hydrogels, gelled synthetic or naturally occurring hydrogels, Collagen I, fibrin, combinations of Collagen I, IV, hyaluronan, chitin, chitosan, alginate, agarose, gelatin, synthetic matrices, biologically inspired synthetic (hybrid) matrices, non-biological gels, chitosan, alginate, agarose and combinations thereof.
